SEQUENCE LISTING (25 sequences)

SEQ ID NO: 1 - RNA, 23 nt, Hepatitis C virus
acggcuagcugugaaugguccgu

SEQ ID NO: 2 - RNA, 23 nt, Hepatitis C virus
ggaccauucacagcuagccguga

SEQ ID NO: 3 - RNA, 23 nt, Homo sapiens
caagagccugagccagcagaucg

SEQ ID NO: 4 - RNA, 23 nt, Homo sapiens
aucugcuggcucaggcucuugag

SEQ ID NO: 5 - RNA, 23 nt, Homo sapiens
ccugugccugccauuacaacugu

SEQ ID NO: 6 - RNA, 23 nt, Homo sapiens
aguuguaauggcaggcacagguc

SEQ ID NO: 7 - DNA, 20 bp, Rattus norvegicus
tccggctcctgctcctctta

SEQ ID NO: 8 - DNA, 23 bp, Rattus norvegicus
ttcttggccatgcgtcaggaggg

SEQ ID NO: 9 - DNA, 24 bp, Rattus norvegicus
gtatgcagctgacttcagggatgt

SEQ ID NO: 10 - DNA, 20 bp, Rattus norvegicus
atccctgcgacccacacaag

SEQ ID NO: 11 - DNA, 21 bp, Rattus norvegicus
ctcccccgccaaccgcaagat

SEQ ID NO: 12 - DNA, 22 bp, Rattus norvegicus
caactgctttggaaggactcgc

SEQ ID NO: 13 - DNA, 25 bp, Rattus norvegicus
ccgatgtatatgcttgcagagttaa

SEQ ID NO: 14 - DNA, 27 bp, Rattus norvegicus
aaccgtcacctgggaccgagacatgta

SEQ ID NO: 15 - DNA, 23 bp, Rattus norvegicus
cagatgattcagagctccataga

SEQ ID NO: 16 - DNA, 25 bp, Homo sapiens
cagaagaactggtacatcagcaaga

SEQ ID NO: 17 - DNA, 27 bp, Homo sapiens
accgatggattccagttcgagtatggc

SEQ ID NO: 18 - DNA, 19 bp, Homo sapiens
gtcagctggatggccacat

SEQ ID NO: 19 - DNA, 17 bp, Homo sapiens
aaccgcaagatcggcgt

SEQ ID NO: 20 - DNA, 22 bp, Homo sapiens
tgcaccgccaaagatggtgctc

SEQ ID NO: 21 - DNA, 19 bp, Homo sapiens
ccgtaccaccgaagatgca

SEQ ID NO: 22 - DNA, 23 bp, Homo sapiens
tgactttgtcacagcccaagata

SEQ ID NO: 23 - DNA, 27 bp, Homo sapiens
tgatgctgcttacatgtctcgatccca

SEQ ID NO: 24 - DNA, 20 bp, Homo sapiens
aatccaaatgcggcatcttc

SEQ ID NO: 25 - protein, 8 aa, Artificial Sequence
  Synthetic construct.
CGRGDSPC

(19) United States
(12) Patent Application Publication
(10) Pub. No.: US 2005/0119202 A1
(43) Pub. Date: Jun. 2, 2005

(54) Medicament to treat a fibrotic disease

(76) Inventors: Roland Kreutzer, Weidenberg (DE); Stefan Limmer, Kulmbach (DE); Detlef Schuppan, Bubenreuth (DE); Matthias John, Hallstadt (DE); Michael Bauer, Erlangen (DE)

Correspondence Address: RANKIN, HILL, PORTER & CLARK LLP, 4080 ERIE STREET, WILLOUGHBY, OH, 44094-7836, US

(21) Appl. No.: 10/493,686

(22) PCT Filed: Oct. 25, 2002
(86) PCT No.: PCT/EP02/11972

(30) Foreign Application Priority Data
Oct. 26, 2001 (DE) 10155280.7

(51) Int. Cl. A61K 48/00; C07H 21/02

(52) U.S. Cl. 514/44; 536/23.1

(57) ABSTRACT

The invention concerns a medicament to treat a fibrotic disease, wherein the medicament contains a double-stranded ribonucleic acid (dsRNA) that is suitable for inhibiting by RNA interference the expression of a gene that is involved in the formation of extracellular matrix.

The invention concerns a medicament and a use to treat a fibrotic disease. It furthermore concerns a double-stranded ribonucleic acid and its use to produce a medicament.

A fibrotic disease is here understood to mean a disease picture characterized by an imbalance between the synthesis of extracellular matrix (ECM) and its breakdown. This imbalance leads to increased formation and deposit of extracellular matrix and connective tissue, respectively. ECM is formed by cells, particularly from collagens, noncollagenous glycoproteins, elastin, proteoglycans, and glycosaminoglycans. The fibrotic disease can, for example, include scar formation after injury of an internal organ or of the skin that exceeds what is required for healing. The excessive formation and deposit of extracellular matrix can lead to functional disturbance or failure of the affected organ, such as the lung, kidney, or liver. ECM is formed in the kidney, for example, by mesangial cells and interstitial fibroblasts. In the liver, hepatic star cells and portal fibroblasts are primarily responsible for the formation of the extracellular matrix. Hepatic star cells, which are normally dormant, can be activated by injury, such as may be the result of toxins or chronic hepatitis. As a consequence they proliferate and transdifferentiate in fibroblasts, which produce an excess of extracellular matrix molecules. Experiments designed to inhibit the synthesis of Type I collagen, an important component of the extracellular matrix, by means of antisense oligonucleotides have led only to a slight inhibition of matrix production. An effective molecular biological method to inhibit matrix production has not been found to date.

A method to inhibit the expression of a target gene in a cell is known from DE 101 00 586 C1, in which an oligoribonucleotide having a double-stranded structure is introduced into the cell. Here one strand of the double-stranded structure is complementary to the target gene.

The task of the present invention is to remove these shortcomings in accordance with the state-of-the-art. In particular, an effective medicament and a use to treat a fibrotic disease is to be made available. Furthermore, a use to produce such a medicament and an active substance that is suitable to inhibit excess formation of extracellular matrix are to be made available.

This task is solved by the features in Claims 1, 21, 22, and 43. Advantageous embodiments result from the features in Claims 2 to 20, 23 to 42, and 44 to 61.

According to the invention, a medicament is intended that contains a double-stranded ribonucleic acid (dsRNA), which is suitable to inhibit by means of RNA interference expression of a gene involved in the formation of extracellular matrix.

A dsRNA is present when the ribonucleic acid, consisting of one or two strands of ribonucleic acid, exhibits a double-stranded structure. Not all nucleotides of a dsRNA must exhibit canonical Watson-Crick base pairs. In particular single, non-complementary base pairs hardly influence effectiveness, if at all. The maximum possible number of base pairs is the number of nucleotides in the shortest strand contained in the dsRNA.

Experiments to treat fibrotic disease by means of antisense oligonucleotides have made it appear that there is little prospect for a molecular biological approach. Surprisingly, however, it has been shown that it is possible to effectively inhibit new formation of connective tissue and ECM, respectively, by means of double-stranded ribonucleic acid. The genes involved in the formation of extracellular matrix are, in terms of the invention, also genes that lead to the formation of factors that cause cells to produce extracellular matrix, or to transform into cells that produce extracellular matrix. Such factors include platelet-derived growth factor (PDGF); transforming growth factor-β (TGF-β), especially TGF-β1, TGF-β2, or TGF-β3; connective tissue growth factor (CTGF); or oncostatin-M. These factors can, for example, initiate and sustain transdifferentiation of hepatic star cells and portal fibroblasts into a phenotype that is similar to myofibroblasts. In contrast to the original cells, this phenotype exhibits an increased proliferation rate and matrix synthesis, often at the same time as reduced breakdown of extracellular matrix (fibrolysis) by matrix-degrading proteases. Liver cells other than hepatic star cells or portal fibroblasts can produce these factors.

In one advantageous embodiment, the gene is a gene that codes for the connective tissue growth factor CTGF; the transforming growth factor-β TGF-β, especially TGF-β1, TGF-β2, or TGF-β3; the Type I or Type II TGF-β receptor; the signal transducers smad-2, smad-3, or smad-4; SARA (smad anchor for receptor activation); PDGF; oncostatin-M, a gene involved in the formation of collagen fibrils; a procollagen; prolyl-4-hydroxylase; lysyl-hydroxylase; lysyl-oxidase; N-propeptidase; or C-propeptidase. Smad-2, smad-3, smad-4, and SARA are involved in the signal transduction triggered by the linkage of TGF-β to the TGF-β Type I or Type II receptor. Prolyl-4-hydroxylase, lysyl-hydroxylase, lysyl-oxidase, N-propeptidase, and C-propeptidase are involved in the formation of collagen fibrils from procollagen, a precursor molecule. N-propeptidase cleaves an N-terminal propeptide and C-propepti-dase cleaves a C-terminal propeptide from a procollagen.

It is particularly advantageous when the procollagen is a procollagen of Type α1(I), α2(I), α1(II), α1(III), α1(V), α2(V), α3(V), α1(VI), α2(VI), α3(VI), α1(XI), α2(XI), or α3(XI). In each case, the Roman numeral in parentheses designates the type of collagen formed from the procollagen. In each case the Arabic numeral designates the chain of the procollagen.

The fibrotic disease may be, for example, a liver fibrosis, fibrosis of the kidney or lung, for example, after an injury, or a formation of scar tissue that exceeds the scar formation required for healing.

Preferably, a strand S1 of dsRNA exhibits a region that is at least segmentally complementary to the gene, consisting, in particular, of fewer than 25 successive nucleotides. “Gene” is here understood to mean the DNA strand of the double-stranded DNA that codes for a protein or peptide, which is complementary to a DNA strand including all transcribed regions that serves as a matrix for transcription. With this gene we are generally dealing with the sense strand. The strand S1 can be complementary to an RNA transcript or its processing product, such as an mRNA, that is formed during the expression of the gene. The protein or peptide is here one that is involved in the formation of extracellular matrix.

The complementary region of the dsRNA can exhibit-in order of ascending preference-19 to 24, 20 to 24, 21 to 23, and particularly 22 or 23 nucleotides. A dsRNA having this structure is particularly efficient in inhibiting the gene. The strand S1 of the dsRNA can exhibit—in order of ascending preference—fewer than 30, fewer than 25, 21 to 24, and particularly 23 nucleotides. The number of these nucleotides is also the maximum possible number of base pairs in the dsRNA.

It has been shown to be particularly advantageous when at least one end of the dsRNA exhibits a single-stranded overhang consisting of 1 to 4, in particular of 2 or 3, nucleotides. In comparison to dsRNA without single-stranded overhangs at at least one end, such dsRNA demonstrates superior effectiveness in inhibiting expression of the gene. Here, one end is a dsRNA region in which a 5′- and a 3′-strand-end is present. DsRNA consisting only of the strand S1 accordingly exhibits a loop structure and only one end. DsRNA consisting of the strand S1 and a strand S2 exhibits two ends. Here, one end is formed in each case by a strand end on the strand S1 and one on the strand S2.

The single-stranded overhang is preferably located at the 3′-end of the strand S1. This location of the single-stranded overhang leads to a further increase in the efficiency of the medicament. In one example, the dsRNA exhibits a single-stranded overhang at only one end, in particular, at the end located at the 3′-end of the strand S1. In dsRNA that exhibits two ends, the other end is blunt, i.e., without overhangs. To enhance the interference action of dsRNA, it has, surprisingly been shown that it is sufficient for dsRNA to have an overhang at one end, without decreasing stability to such an extent as occurs with two overhangs. A dsRNA having only one overhang has proven to be stable enough and particularly effective in a variety of cell culture media, as well as in blood, serum and cells. Inhibition of expression is particularly effective when the overhang is located at the 3′-end of the strand S1.

In addition to the strand S1, the dsRNA preferably exhibits a strand S2, i.e., it is made up of two separate single strands. The medicament is particularly effective when the strand S1 (antisense strand) is 23 nucleotides long, the strand S2 is 21 nucleotides long, and the 3′-end of the strand S1 exhibits a single-stranded overhang consisting of two nucleotides. The dsRNA end that is located at the 5′-end of the strand S1 is blunt. The strand S1 can be complementary to the primary or processed RNA transcript of the gene. Preferably, the dsRNA consists of the strand S2, having Sequence No. 3 and the strand S1, having Sequence No. 4, or of the strand S2, having Sequence No. 5, and the strand S_{1,} having Sequence No. 6, in accordance with the attached sequence listing. Such dsRNA is particularly effective in inhibiting the expression of the gene that codes for Type α1(I) procollagen or CTGF and that is involved in the formation of extra-cellular matrix.

The medicament may exhibit a preparation suitable for inhalation, oral ingestion, infusion or injection, in particular for intravenous or intraperitoneal infusion or injection, or for infusion or injection directly into a tissue affected by the fibrotic disease. A preparation suitable for inhalation, infusion, or injection can most simply consist, in particular exclusively, of the dsRNA and a physiologically tolerated solvent, preferably a physiological saline solution or a physiologically tolerated buffer, in particular a phosphate-buffered saline solution. Surprisingly, it has been shown that dsRNA that has simply been dissolved and administered in such a buffer or solvent is taken up by the cells that express the gene. Expression of the gene, and therefore also the disease, are inhibited without the dsRNA having had to be packaged in a special vehicle. The dsRNA can be present in the medicament in a solution, in particular a physiologically tolerated buffer or a physiological saline solution, surrounded by a micellar structure, preferably a liposome, a capsid, a capsoid, or polymeric nano- or microcapsule, or bound to a polymeric nano- or microcapsule. The physiologically tolerated buffer can be a phosphate-buffered saline solution. A micellar structure, a capsid, capsoid, or polymeric nano- or microcapsule can facilitate uptake of dsRNA in cells that express the gene. The polymeric nano- or microcapsule consists of at least one biologically degradable polymer such as polybutylcyanoacrylate. The polymeric nano- or microcapsule can transport and release in the body dsRNA that is contained in or bound to it.

The dsRNA can be combined with an agent that makes possible the targeted uptake of dsRNA in cells of an organ affected by the fibrotic disease, in particular of the liver, kidney, lung, or skin. Combined means that the dsRNA may be bound to the agent or, as in the case of liposomes or nano- or microcapsules, surrounded by it. Molecules can be embedded in the liposomes or nano- or microcapsules that make possible such targeted uptake, what is called targeting. Preferably, the agent is one that mediates a linkage with a Type VI collagen receptor or the PDGFβ-receptor, in particular of hepatic star cells or myofibroblasts. The hepatic star cells or myofibroblasts can be activated. The cyclical peptide C*GRGDSPC*, in accordance with Sequence No. 25 in the attached sequence listing, is particularly well suited for the Type VI collagen receptor. C* stands for cystein residues, which induce peptide ring formation by means of a disulfide bond.

Preferably, the medicament is present at least in a dosage unit that contains dsRNA in a quantity that makes possible-in order of ascending preference-a maximum dosage of 5 mg, 2.5 mg, 200 μg, 100 μg, 50 μg, and optimally 25 μg per kilogram body weight per day. Surprisingly, it has been shown that dsRNA administered even at this daily dosage exhibits outstanding effectiveness in inhibiting the expression of the gene, and shows anti-fibrotic activity. The dosage unit can be designed for administration or ingestion as a single daily dosage. In this case, the entire daily dose is contained in a single dosage unit. If the dosage unit is designed to be administered or ingested several times per day, the quantity of dsRNA contained in each dose is correspondingly smaller in order to make it possible to achieve the total daily dosage. The dosage unit can also be designed for a single administration or ingestion over several days, e.g., so that the dsRNA is released over several days. The dosage unit then contains a corresponding multiple of the daily dose. The dsRNA is contained in the dosage unit in a sufficient quantity to inhibit the expression of a gene that is involved in the formation of extracellular matrix. The medicament can also be designed such that the sum of several units of the medicament together contain the sufficient quantity. The sufficient quantity can also depend on the pharmaceutical formulation of the dosage unit. To determine what is a sufficient quantity, the dsRNA can be administered in increasing quantities or dosages, respectively. Subsequently, a sample from affected fibrotic tissue can be evaluated using known methods to determine whether inhibition of expression of the aforementioned gene has occurred at this quantity. Such methods may include, e.g., molecular biological, biochemical, or immunological methods.

Furthermore, according to the invention the use of a double-stranded ribonucleic acid to produce a medicament to treat a fibrotic disease is intended, whereby the dsRNA is suitable to inhibit the expression by means of RNA interference of a gene that is involved in the formation of extracellular matrix. Furthermore, according to the invention the use of a double-stranded ribonucleic acid to treat a fibrotic disease is intended, whereby the dsRNA is suitable to inhibit the expression by means of RNA interference of a gene that is involved in the formation of extracellular matrix. Furthermore, a double-stranded ribonucleic acid is intended that is a suitable active agent to inhibit the expression by means of RNA interference of a gene involved in the formation of extracellular matrix in a fibrotic disease.

For further advantageous embodiments of the uses according to the invention and the dsRNA according to the invention, see the previous discussion.

The invention will now be explained exemplary on the basis of graphs. They show:

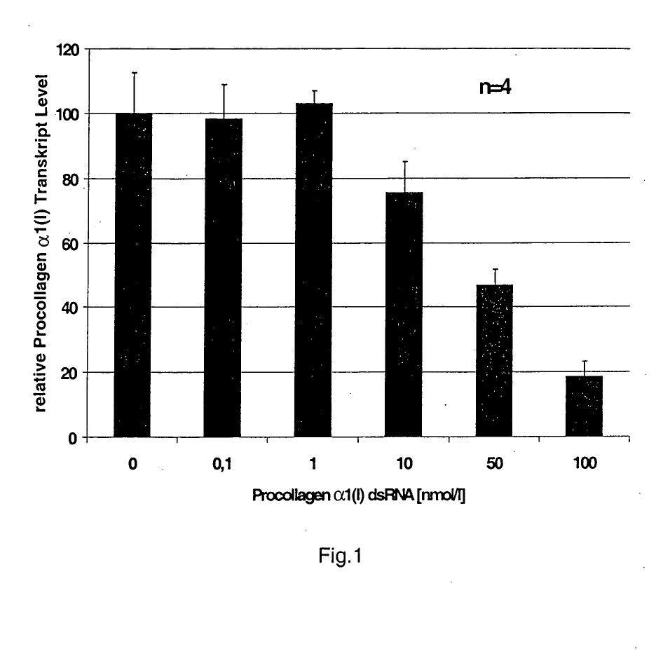

FIG. 1 the relative procollagen-α1(I) transcript levels of RD cells, dependent on the quantity of procollagen-α1(I)-specific dsRNA used in treatment,

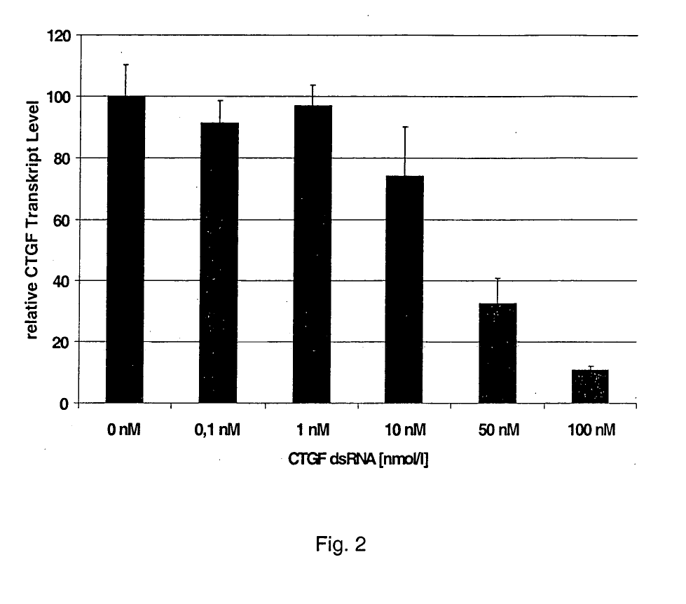

FIG. 2 the relative CTGF transcript levels of RD cells, dependent on the quantity of CTGF-specific dsRNA used in treatment,

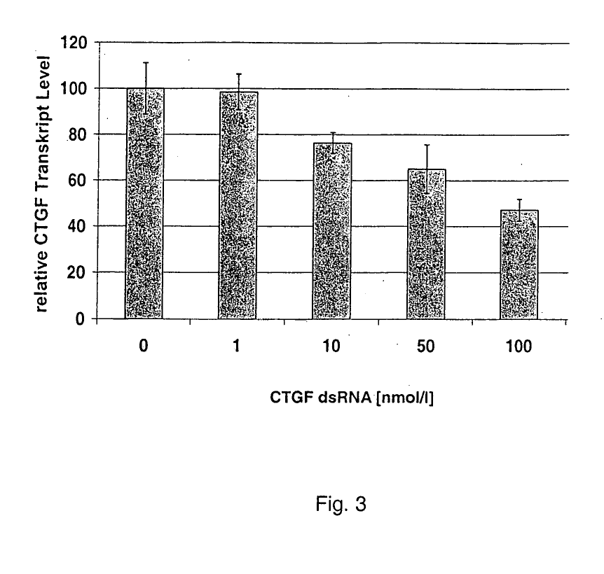

FIG. 3 the relative CTGF transcript levels of CFSC-2G cells, dependent on the quantity of CTGF-specific dsRNA used in treatment, and

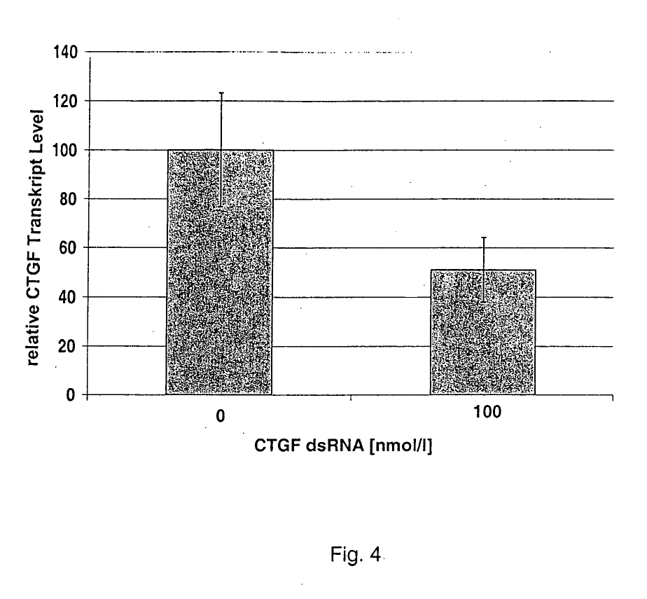

FIG. 4 the relative CTGF transcript levels of hepatic star cells isolated from rats, dependent on the treatment with a CTGF-specific dsRNA.

The following double-stranded oligoribonucleotides having Sequences No. 1 to No. 6, in accordance with the sequence listing, were used for the experiments for transient transfection:

HCV s5/as5, whose strand S1 is complementary to a sequence of the genome of the hepatitis C virus (HCV):

(Sequence No. 1)

S2: 5′- acg gcu agc ugu gaa ugg ucc gu-3′

(Sequence No. 2)

S1: 3′-ag ugc cga ucg aca cuu acc agg -5′

PCA1+2, whose strand S1 is complementary to a sequence of the human procollagen α1(I) gene, and the procollagen α1(I) gene from Rattus norvegicus that is in this region to the 100%-homologous to it:

(Sequence No. 3)

S2: 5′- caa gag ccu gag cca gca gau cg-3′

(Sequence No. 4)

S1: 3′-ga guu cuc gga cuc ggu cgu cua -5′

CTG1+2, whose strand S1 is complementary to a sequence of the human CTGF gene and the CTGF gene from Rattus norvegicus that is in this region to the 100%-homologous to it:

(Sequence No. 5)

S2: 5′- ccu gug ccu gcc auu aca acu gu-3′

(Sequence No. 6)

S1: 3′-cu gga cac gga cgg uaa ugu uga -5′

The following cells were used for the experiments:
RD cells: these are cells of a human embryonic rhabdomyosarcoma cell line. This cell line may be obtained under No. CCL136 from the American Type Culture Collection (ATCC), P.O. Box 1549, Manassas, Va. 20108, USA.
CFSC-2G cells: these are cells from a rat hepatic star cell line that was made available by Dr. Marcos Rojkind (Liver Research Center, Albert Einstein College of Medicine, Bronx, New York City, N.Y., USA). The isolation of the CFSC stem cells is described in: Laboratory Investigation 65 (1991), 644-53. The isolation and characterization of the. CFSC-2G subclone is described in: Patricia Greenwel et al., Laboratory Investigation 69 (1993), 210-26.
Primary hepatic star cells isolated from rat liver, in accordance with Knook, D. et al., Exp. Cell Res. 139 (1982), pages 468 to 471.

All cells were cultured in Dulbecco's Modified Eagle's Medium (DMEM) with 862 mg/l 1-alanyl-1-glutamine and 4.5 g/l glucose (Invitrogen GmbH, Technology Park Karlsruhe, Emmy-Noether Strasse 10, D-76131 Karlsruhe), with the addition of 10% heat-deactivated fetal calf serum (FCS), 100 IU/ml penicillin and 100 μg/ml streptomycin (cell culture medium). Culturing was done in an incubator at 37° C. in a moist atmosphere of 8% CO₂ and 92% air.

Transient transfection of RD cells with dsRNA was achieved by lipofection with DNA-laden liposomes from cationic lipids. The Lipofectamine Plus reagent kit from Invitrogen was used for that purpose. It contains a lipofectamine- and a plus reagent. Each transfection was done 4 times in parallel in accordance with manufacturer instructions. For a transfection, approximately 70,000 RD cells/well were seeded in a sterile 12-well plate. Twenty-four hours later, 5 μl of a 20 μmol/l aqueous solution containing the respective dsRNA was diluted in 100 μl DMEM per 2 wells in a 12-well plate. To this was added in each case 10 μl Plus reagent, mixed, and incubated for 15 minutes at room temperature. Next, 100 μl of a fresh 1:25 dilution of lipofectamine reagent in DMEM (corresponding to 240 μg lipid mixture/ml) was added, mixed, and the formation of DNA-laden liposomes was made possible by incubation for 15 minutes at RT. After that, the cell culture medium was drawn off from the cells, and the cells were washed twice each with 1 ml DMEM per well. Each transfection assay was diluted with 1 ml DMEM, and 0.6 ml/well of this was pipetted onto the cells (2 wells per assay). After incubating for 4 hours in an incubator, 1 ml of cell culture medium was added to each well and incubated for another 44 hours.

For transient transfection of hepatic star cells and CFSC-2G cells, dsRNA was introduced into the cells by means of oligofectamine (Invitrogen). For this, CFSC-2G or hepatic star cells isolated from rats was seeded at a density of 20,000 cells/well in a sterile 12-well plate. Twenty-four hours after seeding, 4 μl oligofectamine was diluted in 11 μl DMEM per assay, and incubated at room temperature for 10 minutes. Furthermore, 5 μl of a 20 mol/l aqueous solution containing dsRNA was diluted in 185 μl DMEM per assay (2 wells of a 12-well plate). 15 μl each of the prediluted oligofectamine was pipetted into the diluted dsRNA, mixed, and incubated for 20 minutes at room temperature. Finally, 1050 μl DMEM was added to the assays. 600 μl each of the resulting mixture was added to the cells after they had been washed twice with 1 ml DMEM per well. After incubation for 4 hours in the incubator, 1 ml of cell culture medium was added to each well and incubated for 44 hours in the incubator.

The action of the dsRNA on the transcript levels of genes involved in the formation of extracellular matrix was determined in all the cells studied by means of quantitative PCR. After 44 hours in an incubator, the cells were lysed, and the RNA they contained was isolated using the PeqGold RNAPure kit (PEQLAB Biotechnologie GmbH, Carl-Thiersch-Str. 2b, D-91052 Erlangen, Order No. 30-1010) in accordance with manufacturer instructions.

cDNA was formed in each case by using the same quantities of RNA (100-1000 ng) for reverse transcription, using Superscript II (Invitrogen GmbH, Technology Park, Karlsruhe, Emmy-Noether Strasse 10, D-76131 Karlsruhe; Catalogue No. 18064-014). 100 pmol oligo-dT primer and 50 pmol random primer were used as the primers. 10 μl of RNA (100-1000 ng), 0.5 μl oligo-dT primer (100 pmol), and 1 μl random primer (50 pmol) were incubated for 10 minutes at 70° C., and then stored on ice for short time. Subsequently, 7 μl reverse transcriptase mix (4 μl of 5×buffer; 2 μl of 0.1 mol/l DTT; 1 Al each of 10 mmol/l DNTP), 1 μl Superscript II, and 1 μl of the ribonuclease inhibitor RNAsin® (Promega GmbH, Schildkrötstr. 15, D-68199 Mannheim) were added. The mixture was then kept at 25° C. for 10 minutes, then at 42° C. for 1 hour, and finally at 70° C. for 15 minutes.

The action of dsRNA in cells transfected with it on the expression of the genes that code for procollagen α1(I) and CTGF was demonstrated by determining the quantity of transcript (transcript levels) of these genes by means of quantitative “real-time” RT-PCR. For this, specific cDNA quantities from the same volumes of formed cDNA were quantified in a “Light-Cycler” (Roche Diagnostics GmbH) in accordance with the “TaqMan” method (PerkinElmer, Ferdinand-Porsche-Ring 17, D-63110 Rodgau-Jügesheim) in accordance with manufacturer instructions, using the LightCycler Fast Start DNA Master Hybridization Probes kit (Roche Diagnostics GmbH). Detection was done with a probe marked at the 5′-end with fluorophore 6′-FAM (carboxyfluoresceine), and at the 3′-end with the quencher molecule TAMRA (carboxy-tetra-methyl-rhodamine). The fluorophore is excited by light. It transfers the excitation energy to the 3′-sided quencher molecule that is in the immediate vicinity. During the extension phases of PCR, the 5′-3′ exonuclease activity of Taq DNA polymerase leads to hydrolysis of the probe, and thus also to a spatial separation of fluorophore from the quencher molecule. Fluorescence of 6′-FAM is progressively less quenched. It therefore increases and is quantitatively determined. Quantification is done with a standard curve made up using known transcript quantities or a dilution series of a reference cDNA. Furthermore, the transcript level of the housekeeping gene β2-microglobulin was determined and used for standardization. β2-microglobulin is a protein that is expressed constitutively in a constant quantity. The quantity of procollagen α1(I)- or CTGF-cDNA was determined as a ratio to the quantity of β2-microglobulin-CDNA, and is shown graphically in FIGS. 1 to 4 as the relative transcript level.

The following primers and Taqman probes were used to determine the transcript levels in rat cells of procollagen α1(I) and CTGF by means of real-time RT-PCR:

| Target | | TaqMan probe with | |
|---|---|---|---|
| molecule | 5′ Primer | 5′FAM + 3′-TAMRA | 3′ Primer |
| Procollagen | TCCGGCTCCTGCTCCTCTTA | TTCTTGGCCATGCGTCAGGAGGG | GTATGCAGCTGACTTCAGGGATGT |
| α1(I) | | | |
| CTGF | ATCCCTGCGACCCACACAAG | CTCCCCCGCCAACCGCAAGAT | CAACTGCTTTGGAAGGACTCGC |
| β2- | CCGATGTATATGCTTGCAGAGTTAA | AACCGTCACCTGGGACCGAGACATGTA | CAGATGATTCAGAGCTCCATAGA |
| microglobulin | | | |

The following primers and TaqMan probes were used to determine the transcript levels in human cells of procollagen α1(I) and CTGF by means of real-time RT-PCR:

| Target | | TaqMan probe with | |
|---|---|---|---|
| molecule | 5′ Primer | 5′-FAM + 3′-TAMRA | 3′ Primer |
| Procollagen | CAGAAGAACTGGTACATCAGCAAGA | ACCGATGGATTCCAGTTCGAGTATGGC | GTCAGCTGGATGGCCACAT |
| α1(I) | | | |
| CTGF | AACCGCAAGATCGGCGT | TGCACCGCCAAAGATGGTGCTC | CCGTACCACCGAAGATGCA |
| β2- | TGACTTTGTCACAGCCCAAGATA | TGATGCTGCTTACATGTCTCGATCCCA | AATCCAAATGCGGCATCTTC |
| microglobulin | | | |

FIGS. 1 to 4 show the action of dsRNA. In order to guarantee constant transfection efficiency in the experiments, all cells were transfected with 100 nmol/l dsRNA. For this, 0 to 100 nmol/l of specific dsRNA directed against procollagen α1(I) or CTGF was completed with the nonspecific HCV s5/as5 dsRNA to a concentration of 100 nmol/l, and transfected in cells. The transcript level measured with the 0 nmol/l specific dsRNA was arbitrarily defined as 100%.

The results for RD cells that were transfected with increasing concentrations of dsRNA directed against procollagen α1(I) are shown in FIG. 1. The action of dsRNA is dependent on concentration. The procollagen α1(I) transcript level could be reduced to 20% with 100 nmol/l PCA1+2 dsRNA. Expression of β2-microglobulin was not changed by the dsRNA. This demonstrates the specificity of the dsRNA used.

FIG. 2 shows the relative transcript levels of the CTGF gene dependent on the concentration of the CTG1+2 dsRNA used for transfection. Here, too, the effect of the dsRNA used is dependent on concentration. 100 nmol/l CTG1+2 dsRNA reduces the transcript level to 10%, while 50 nmol in dsRNA lowers the transcript level to 32% of that of cells treated with nonspecific HCV s5/as5 dsRNA. Here, too, the expression of β2-microglobulin is unchanged.

FIG. 3 shows the relative transcript levels of the CTGF gene in CFSC-2G cells 48 hours after transfection. Here, too, there is a concentration-dependent reduction in transcript levels by the dsRNA that is used.

FIG. 4 shows the relative transcript levels of the CTGF gene in hepatic star cells and myofibroblasts, respectively, isolated from rats. The cells were cultured for 7 days on plastic. As a result they were already activated. 48 hours after transfection with 100 nmol/l dsRNA, there was an approximately 50% reduction in transcription.

1-61. (canceled)

62. Medicament to treat a fibrotic disease, wherein the medicament contains a double-stranded ribonucleic acid (dsRNA) that is suitable to inhibit by means of RNA interference expression of a gene that is involved in the formation of extracellular matrix, whereby the medicament exhibits a preparation consisting exclusively of the dsRNA and a physiologically tolerated solvent.

63. Medicament in accordance with claim 62, wherein the gene is a gene that codes for CTGF, TGF-β, the Type I or Type II TGF-β receptor, smad-2, smad-3, or smad-4, SARA, PDGF, oncostatin-M, a gene involved in the formation of collagen fibrils, a procollagen, prolyl-4-hydroxylase, lysyl-hydroxylase, lysyloxidase, N-propeptidase, or C-propeptidase.

64. Medicament in accordance with claim 63, wherein the procollagen is of Type α1(I), α2(I), α1(II), α1(III), α1(V), α2(V), α3(V), α1(VI), α2(VI), α3(VI), α1(XI), α2(XI), or α3(XI).

65. Medicament in accordance with claim 62, wherein the fibrotic disease is a liver fibrosis, fibrosis of the kidney or lung, or the formation of scar tissue that exceeds the scar formation necessary for healing.

66. Medicament in accordance with claim 62, wherein a strand S1 of dsRNA exhibits a region consisting in particular of fewer than 25 successive nucleotides that is at least segmentally complementary to the gene.

67. Medicament in accordance with claim 62, wherein the complementary region exhibits 19 to 24, preferably 20 to 24, especially preferably 21 to 23, in particular 22 or 23 nucleotides.

68. Medicament in accordance with claim 62, wherein the strand S1 exhibits fewer than 30, preferably fewer than 25, particularly preferably 21 to 24, in particular 23 nucleotides.

69. Medicament in accordance with claim 62, wherein at least one end of the dsRNA exhibits a single-stranded overhang, consisting of 1 to 4, in particular 2 or 3 nucleotides.

70. Medicament in accordance with claim 69, wherein the single-stranded overhang is located at the 3′-end of the strand S1.

71. Medicament in accordance with claim 62, wherein the dsRNA exhibits a single-stranded overhang at only one end, in particular at the end located at the 3′-end of the strand S1.

72. Medicament in accordance with claim 62, wherein the dsRNA exhibits a strand S2 in addition to the strand S1.

73. Medicament in accordance with claim 72, wherein the strand S1 is 23 nucleotides long, the strand S2 is 21 nucleotides long, and the 3′-end of the strand S1 exhibits a single-stranded overhang consisting of two nucleotides, while the dsRNA end that is located at the 5′-end of the strand S1 is blunt.

74. Medicament in accordance with claim 62, wherein the strand S1 is complementary to the primary or processed RNA transcript of the gene.

75. Medicament in accordance with claim 62, wherein the dsRNA consists of the strand S2 having Sequence No. 3 and the strand S1 having Sequence No. 4, or of the strand S2 having the Sequence No. 5 and the strand S1 having Sequence No. 6 in accordance with the attached sequence listing.

76. Medicament in accordance with claim 62, wherein the medicament exhibits a preparation suitable for inhalation, infusion or injection, in particular for intravenous or intraperitoneal infusion or injection, or for infusion or injection directly into a tissue affected by the fibrotic disease.

77. Medicament in accordance with claim 62, wherein the medicament is present at least in a dosage unit that contains dsRNA in a quantity that makes possible—in order of ascending preference—a maximum dosage of 5 mg, 2.5 mg, 200 μg, 100 μg, 50 μg, and optimally 25 pg per kilogram body weight per day.

78. Use of a double-stranded ribonucleic acid (dsRNA) to produce a medicament to treat a fibrotic disease, wherein the dsRNA is suitable to inhibit by RNA interference the expression of a gene that is involved in the formation of extracellular matrix, wherein the dsRNA is contained in a preparation consisting exclusively of the dsRNA and a physiologically tolerated solvent.

79. Use of a double-stranded ribonucleic acid (dsRNA) to treat a fibrotic disease, wherein the dsRNA is suitable to inhibit by RNA interference the expression of a gene that is involved in the formation of extracellular matrix, wherein the dsRNA is contained in a preparation consisting exclusively of the dsRNA and a physiologically tolerated solvent.

80. Use in accordance with claim 78, wherein the gene is a gene that codes for CTGF, TGF-β, the Type I or Type II TGF-β receptor, smad-2, smad-3, or smad-4, SARA, PDGF, oncostatin-M, a gene involved in the formation of collagen fibrils, a procollagen, prolyl-4-hydroxylase, lysyl-hydroxylase, lysyl-oxidase, N-propeptidase, or C-propeptidase.

81. Use in accordance with claim 80, wherein the procollagen is of Type α1(I), α2(I), α1(II), α1(III), α1(V), α2(V), α3(V), α1(VI), α2(V), α3(VI), α1(XI), α2(XI), or α3(XI).

82. Use in accordance with claim 78, wherein the fibrotic disease is a liver fibrosis, fibrosis of the kidney or lung, or the formation of scar tissue that exceeds the scar formation necessary for healing.

83. Use in accordance with claim 78, wherein a strand S1 of dsRNA exhibits a region consisting in particular of fewer than 25 successive nucleotides that is at least segmentally complementary to the gene.

84. Use in accordance with claim 78, wherein the complementary region exhibits 19 to 24, preferably 20 to 24, especially preferably 21 to 23, in particular 22 or 23 nucleotides.

85. Use in accordance with claim 78, wherein the strand S1 exhibits fewer than 30, preferably fewer than 25, particularly preferably 21 to 24, in particular 23 nucleotides.

86. Use in accordance with claim 78, wherein at least one end of the dsRNA exhibits a single-stranded overhang, consisting of 1 to 4, in particular 2 or 3 nucleotides.

87. Use in accordance with claim 86, wherein the single-stranded overhang is located at the 3′-end of the strand S1.

88. Use in accordance with claim 78, wherein the dsRNA exhibits a single-stranded overhang at only one end, in particular at the end located at the 3′-end of the strand S1.

89. Use in accordance with claim 78, wherein the dsRNA exhibits a strand S2 in addition to the strand S1.

90. Use in accordance with claim 89, wherein the strand S1 is 23 nucleotides long, the strand S2 is 21 nucleotides long, and the 3′-end of the strand S1 exhibits a single-stranded overhang consisting of two nucleotides, while the dsRNA end that is located at the 5′-end of the strand S1 is blunt.

91. Use in accordance with claim 78, wherein the strand S1 is complementary to the primary or processed RNA transcript of the gene.

92. Use in accordance with claim 78, wherein the dsRNA consists of the strand S2 having Sequence No. 3 and the strand S1 having Sequence No. 4, or of the strand S2 having the Sequence No. 5 and the strand S1 having Sequence No. 6 in accordance with the attached sequence listing.

93. Use in accordance with claim 78, wherein the dsRNA is present in a preparation suitable for inhalation, infusion or injection, in particular for intravenous or intraperitoneal infusion or injection or for infusion or injection directly into a tissue affected by the fibrotic disease.

94. Use in accordance with claim 78, wherein the dsRNA is administered by means of inhalation, infusion, or injection, in particular by intravenous or intraperitoneal infusion or injection, or infusion or injection directly into tissue affected by the fibrotic disease.

95. Use in accordance with claim 78, wherein the dsRNA is used—in order of ascending preference—in a maximum dosage of 5 mg, 2.5 mg, 200 μg, 100 μg, 50 μg, and optimally 25 μg per kilogram body weight per day.

96. Double-stranded ribonucleic acid (dsRNA) that is suitable to inhibit by RNA interference the expression of a gene that is involved in the formation of extracellular matrix in a fibrotic disease, whereby the dsRNA is contained in a preparation consisting exclusively of the dsRNA and a physiologically tolerated solvent.

97. DsRNA in accordance with claim 96, wherein the gene is a gene that codes for CTGF, TGF-β, the Type I or Type II TGF-β receptor, smad-2, smad-3, or smad-4, SARA, PDGF, oncostatin-M, a gene involved in the formation of collagen fibrils, a procollagen, prolyl-4-hydroxylase, lysyl-hydroxylase, lysyl-oxidase, N-propeptidase, or C-propeptidase.

98. DsRNA in accordance with claim 97, wherein the procollagen is of Type α1(I), α2(I), α1(II), α1(III), α1(V), α2(V), α3(V), α1(VI), α2(VI), α3(VI), α1(XI), α2(XI), or α3(XI).

99. DsRNA in accordance with claim 96, wherein the fibrotic disease is a liver fibrosis, fibrosis of the kidney or lung, or unwanted scar formation.

100. DsRNA in accordance with claim 96, wherein a strand S1 of dsRNA exhibits a region consisting in particular of fewer than 25 successive nucleotides that is at least segmentally complementary to the gene.

101. DsRNA in accordance with claim 96, wherein the complementary region exhibits 19 to 24, preferably 20 to 24, especially preferably 21 to 23, in particular 22 or 23 nucleotides.

102. DsRNA in accordance with claim 96, wherein the strand S1 exhibits fewer than 30, preferably fewer than 25, particularly preferably 21 to 24, in particular 23 nucleotides.

103. DSRNA in accordance with claim 96, wherein at least one end of the dsRNA exhibits a single-stranded overhang, consisting of 1 to 4, in particular 2 or 3 nucleotides.

104. DsRNA in accordance with claim 103, wherein the single-stranded overhang is located at the 3′-end of the strand S1.

105. DsRNA in accordance with claim 96, wherein the dsRNA exhibits a single-stranded overhang at only one end, in particular at the end located at the 3′-end of the strand S1.

106. DsRNA in accordance with claim 96, wherein the dsRNA exhibits a strand S2 in addition to the strand S1.

107. DsRNA in accordance with claim 106, wherein the strand S1 is 23 nucleotides long, the strand S2 is 21 nucleotides long, and the 3′-end of the strand S1 exhibits a single-stranded overhang consisting of two nucleotides, while the dsRNA end that is located at the 5′-end of the strand S1 is blunt.

108. DsRNA in accordance with claim 96, wherein the strand S1 is complementary to the primary or processed RNA transcript of the gene.

109. DsRNA in accordance with claim 96, wherein the dsRNA consists of the strand S2 having Sequence No. 3 and the strand S1 having Sequence No. 4, or of the strand S2 having the Sequence No. 5 and the strand S1 having Sequence No. 6 in accordance with the attached sequence listing.

110. DsRNA in accordance with claim 96, wherein the dsRNA is present in a preparation suitable for inhalation, infusion or injection, in particular for intravenous or intraperitoneal infusion or injection or for infusion or injection directly into a tissue affected by the fibrotic disease.

111. DsRNA in accordance with claim 104, wherein at least one end of the dsRNA exhibits a single-stranded overhang, consisting of 1 to 4, in particular 2 or 3 nucleotides.

112. DsRNA in accordance with claim 111, wherein the single-stranded overhang is located at the 3′-end of the strand S1.

113. DsRNA in accordance with claim 104, wherein the dsRNA exhibits a single-stranded overhang at only one end, in particular at the end located at the 3′-end of the strand S1.

114. DsRNA in accordance with claim 104, wherein the dsRNA exhibits a strand S2 in addition to the strand S1.

115. DsRNA in accordance with claim 114, wherein the strand S1 is 23 nucleotides long, the strand S2 is 21 nucleotides long, and the 3′-end of the strand S1 exhibits a single-stranded overhang consisting of two nucleotides, while the dsRNA end that is located at the 5′-end of the strand S1 is blunt.

116. DsRNA in accordance with claim 104, wherein the strand S1 is complementary to the primary or processed RNA transcript of the gene.

117. DsRNA in accordance with claim 104, wherein the dsRNA consists of the strand S2 having Sequence No. 3 and the strand S1 having Sequence No. 4, or of the strand S2 having the Sequence No. 5 and the strand S1 having Sequence No. 6 in accordance with the attached sequence listing.

118. DsRNA in accordance with claim 104, wherein the dsRNA is present in a preparation suitable for inhalation, oral ingestion, infusion or injection, in particular for intravenous or intraperitoneal infusion or injection or for infusion or injection directly into a tissue affected by the fibrotic disease.

119. DsRNA in accordance with claim 104, wherein the dsRNA is present in a solution, in particular a physiologically tolerated buffer or a physiological saline solution, surrounded by a micellar structure, preferably a liposome, capsid, capsoid, or a polymeric nano- or microcapsule, or bound to a polymeric nano- or microcapsule.

120. DsRNA in accordance with claim 104, wherein the dsRNA is combined with an agent that makes possible a targeted uptake of the dsRNA in cells of an organ affected by fibrotic disease, in particular of the liver, kidney, lung, or skin.

121. DsRNA in accordance with claim 120, wherein the agent is one that mediates a linkage with a Type VI collagen receptor or the PDGFβ-receptor, in particular of hepatic star cells or myofibroblasts.

122. DsRNA in accordance with claim 121, wherein the agent is the cyclical peptide C*GRGDSPC*.
